(19) United States
(12) Patent Application Publication
(10) Pub. No.: US 2006/0199024 A1
(43) Pub. Date: Sep. 7, 2006

(54) Biocompatible thermal spray coating made from a nanostructured feedstock

(76) Inventors: Rogerio Soares Lima, Boucherville (CA); Basil Richard Marple, Boucherville (CA); Hua Li, Singapore (SG); Khiam Aik Khor, Singapore (SG)

Correspondence Address: LAUBSCHER & LAUBSCHER, P.C., 1160 SPA ROAD, SUITE 2B, ANNAPOLIS, MD, 21403, US

(21) Appl. No.: 11/363,612

(22) Filed: Feb. 28, 2006

(30) Foreign Application Priority Data
Mar. 1, 2005 (CA) 2,499,202

(51) Int. Cl. A61L 33/00; B05D 1/08; B32B 15/04; B32B 9/00; B32B 19/00

(52) U.S. Cl. 428/469; 427/2.1; 427/446; 428/472; 428/701; 428/702

(57) ABSTRACT

A method of making a biocompatible coating for an implant involves thermally spraying a feedstock of nanostructured agglomerated particles of a biocompatible material onto a substrate, and controlling the spray parameters such that the agglomerated particles strike the substrate as a mix of fully molten and semi-molten particles and the semi-molten particles become distributed throughout the coating.

### FIELD OF THE INVENTION

This invention relates to the field of prosthetics, and in particular to a method of making biocompatible implants with improved mechanical and osseointegration properties, and to novel coatings for such implants.

### BACKGROUND OF THE INVENTION

Metallic implants, such as hip joints, are used to replace body parts that have become worn out. Such implants are normally made of titanium alloys (Ti-6A1-4V). These alloys exhibit high mechanical strength and cause no harm to the human body. They are bioinert. However, due to their lack of biointeraction, they do not form strong bonds between the metal surface and the bone cells, known as osteoblasts. Another agent must be employed to assist in the attachment of the metallic implant to the osteoblast cells or osseointegration as it is known.

One way to promote osseointegration and bonding between the implant and the surrounding bone is the use of a biocompatible coating, such as hydroxyapatite (HA), that is well bonded to the surface of the implant (prosthetic device). This coating is normally applied by a thermal spray technique prior to implantation in the body. Following implantation the osteoblast cells attach to the biocompatible coating, thereby providing an increased rate of apposition and bonding.

Despite the success of HA coatings there are still drawbacks. HA thermal spray coatings exhibit poor mechanical performance, with bond strength values on Ti6A1-4V substrates (testing method ASTM C633) normally equal to or below 31 MPa (H. Li, K. A. Khor, P. Cheang, “Effect of Powders’ Melting State on the Properties of HVOF Sprayed Hydroxyapatite Coatings”, Materials Science and Engineering A, 293 (2000) 71-80). The long term stability of HA thermal spray coatings when implanted in the body is also a concern. It has been observed that HA coatings in contact with human tissue may dissolve and become detached, exposing the implant3 s metallic surface and thereby causing adverse effects on interfacial bone apposition to the implant and on its mechanical stability. HA coatings also suffer higher degrees of degradation when submitted to loading, which is an unwanted characteristic for a coating on an implant. Another important complication of HA thermal spray coatings is the formation of debris. It has been suggested that these HA particulate chips may contribute to the accelerated wear of metal-on-polyethylene articulation in hip joint implants.

The longevity of orthopedic implants ranges from only about 12 to 15 years. Thus, the majority of patients that receive a hip replacement at age 65 or below will require at least one revision surgery. In 1997, 13% of hip arthroplasties were revisions of previously failed hip replacements. The current life expectancy of orthopedic prostheses is a serious problem that results in increased costs and discomfort for the patient.

Additional information pertaining to the state of the art can be found in the following references: O. Reikeras, R. B. Gunderson, “Failure of HA Coating on a Gritblasted Acetabular Cup”, Acta Orthop. Scand., 73(1), 2002, 104-108; K. A. Lai, W. J. Shen, C. H. Chen, C. Y. Yang, W. P. Hu, G. L. Chang, “Failure of Hydroxyapatite-Coated Acetabular Cups”, The Journal of Bone & Joint Surgery (Br), 84-B(5), 2002, 641-646; T. J. Webster, C. Ergun, R. H. Doremus, R. W. Siegel, R. Bizios, “Specific Proteins Mediate Enhanced Osteoblast Adhesion on Nanophase Ceramics”, Journal of Biomedical Materials Research, 51(3), 2000, 475-483; Lima, B. R. Marple, “Enhanced Ductility in Thermally Sprayed Titania Coating Synthesized using a Nanostructured Feedstock”, Materials Science and Engineering A, 395, 2005, 269-280; and U.S. Pat. No. 6,835,449.

### SUMMARY OF THE INVENTION

There is disclosed herein a method of making new coating arising with enhanced mechanical properties and biocompatibility.

According to the present invention there is provided a method of making a biocompatible implant, comprising providing a feedstock of nanostructured agglomerated particles of a biocompatible material; thermally spraying said particles onto a substrate to form a coating; and controlling the spray parameters such that said agglomerated particles strike the substrate as a mix of fully molten and semi-molten particles and the semi-molten particles become distributed throughout the coating.

In accordance with embodiments of the invention the HA thermal spray coatings are replaced with a new coating, which has a longer life for implants and exhibits the following characteristics: (i) non-toxic to and non-absorbable by the human body, (ii) superior mechanical performance when compared to HA thermal spray coatings; and (iii) good biocompatibility with osteoblast cells.

The coating is preferably made from thermally sprayed nanostructured agglomerated titania (TiO₂) particles, although other materials, such as hydroxyapatite, zirconia, and alumina (or a combination of these) may be used for the nanostructured feedstock.

The specific particle size (diameter) distribution preferably lies in the range from about 0.1 to about 200 microns. The particles are preferably applied using a high velocity oxy-fuel (HVOF) thermal spray torch. However, other processes such as air plasma spray (APS), vacuum plasma spray (VPS), low pressure plasma spray (LPPS), high velocity air-fuel (HVAF), high frequency pulse detonation (HFPD), detonation gun, suspension plasma spray and suspension HVOF spray may be employed. When compared to other titania coatings made from nanostructured or conventional feedstock powders, the high velocity oxy-fuel (HVOF) sprayed titania coatings made from a nanostructured feedstock exhibit: (i) superior abrasion resistance, (ii) superior slurry-erosion resistance at 30°, (iii) superior slurry-erosion resistance at 90°, (iv) superior bond strength, (v) isotropic characteristics, (vi) superior toughness and (vii) enhanced ductility.

In accordance with another aspect of the invention there is provided a biocompatible implant comprising a substrate, and a thermally applied coating on said substrate for promoting osteoblast growth, said applied coating including a proportion of agglomerated nanostructured feedstock particles retaining their original nanostructure distributed throughout said coating.

The nanostructural characteristics are preferably formed from semi-molten agglomerates preferably having a diameter from about 0.1 to about 200 microns containing particles smaller than 100 nm, although individual nanoparticles having diameters varying from 100 to 300 nm can be distributed throughout the coating.

### BRIEF DESCRIPTION OF THE DRAWINGS

The invention will now be described in more detail, by way of example only, with reference to the accompanying drawings, in which:

FIG. 1 is a SEM picture showing the growth and proliferation of human osteoblast cells on the surface of a titania coating made in accordance with an embodiment of the invention after a three day incubation period;

FIG. 2 is a SEM picture of osteoblast cells (obtained from rat calvaria) cultured for 7 days on the surface of the titania coating made from the nanostructured feedstock;

FIG. 3 is a SEM picture of osteoblast cells (obtained from rat calvaria) cultured for 7 days on the surface of the APS HA coating;

FIG. 4 shows osteoblast cells stained for alkaline phosphatase activity (shown in red) after 15-day culture on the surface of the titania coating made from the nanostructured feedstock;

FIG. 5 shows osteoblast cells (obtained from rat calvaria) stained for alkaline phosphatase activity (shown in red) after 15-day culture on the surface of the APS HA coating; and

FIG. 6 shows the relative intensity of red staining for the osteoblast cells (obtained from rat calvaria) on the surface of the titania coating made from the nanostructured feedstock and APS HA coating after a 15-day cell culture; and

FIG. 7 shows an exemplary embodiment of a hip joint with a coating in accordance with an embodiment of the invention.

### DETAILED DESCRIPTION OF THE PREFERRED EMBODIMENTS

Nanophase ceramics, such as nanostructured titania, exhibit enhanced osteoblast adhesion and proliferation when compared to conventional ceramics. Osteoblast cells do not grow and proliferate directly on the surface of these ceramics. Instead, before the osteoblast growth and attachment, proteins such as vitronectin and fibronectin have to be adsorbed by the ceramic surface (T. J. Webster, C. Ergun, R. H. Doremus, R. W. Siegel, R. Bizios, “Specific Proteins Mediate Enhanced Osteoblast Adhesion on Nanophase Ceramics”, Journal of Biomedical Materials Research, 51(3), 2000, 475-483-K. Anselme, “Osteoblast Adhesion on Biomaterials”, Biomaterials, 21, 2000, 667-681). These proteins are believed to be the key agents for osteoblast adhesion. The proteins exhibit lengths in the order of nanometers. In order to be adsorbed they must encounter surfaces with nanostructural characteristics like nanoprotuberances, nanoirregularities and nanopores. These characteristics (i.e., nanoprotuberances, nanoirregularities and nanopores) are found in nanostructured ceramics.

In accordance with the principles of the invention, it has been found that when titania (TiO₂), a material that is non-toxic and non-absorbable by the human body, is produced as a nanostructured feedstock, and then deposited via the thermal spray process, employing spray conditions at which feedstock particles reach a relatively high velocity and an intermediate temperature (allowing retention of some of the nanophase), the resulting coating has a superior mechanical performance (when compared to HA thermal spray coatings) and good biocompatibility with the osteoblast cells.

To produce a thermal spray coating in accordance with the principles of the invention, the following steps are carried out: Agglomerated particles of titania exhibiting a particle size within the range from about 0.1 to about 200 microns, with each agglomerate comprised of a large number of individual nanostructured particles of titania smaller than 100 nm, (although the individual particle size can go as high as 300 nm) are placed in a powder feeder (or any other type of feeding machine) that provides a continuous flow of particles (from 1 to 100 g/min.) into the spray jet of a thermal spray torch. Instead of titania, it is possible to use hydroxyapatite, zirconia, and alumina (or a combination thereof).

The spray jet of the thermal spray torch can be produced by a plasma or combustion process. The plasma gases can be argon, hydrogen, nitrogen and helium or a combination thereof. The flame of the thermal spray torch can conveniently be formed by the combustion of oxygen and propylene, oxygen and hydrogen, oxygen and methane (natural gas), oxygen and acetylene, or oxygen and propane. Alternatively, air can be used to replace oxygen for the combustion reactions.

The substrate is preferably placed at about 1-100 cm from the exit of the thermal spray torch nozzle.

The plasma or the flame of the thermal spray torch preferably serves two purposes: (i) to melt totally or partially the agglomerates and (ii) to propel the agglomerates (in a spray jet) towards the substrate structure. The average temperature and velocity of the sprayed particles at the substrate position is preferably 1500-3000° C. and 100-1000 m/s, respectively. The coating is formed by the successive impact, overlapping and interlocking of the fully molten and semi-molten sprayed particles on the substrate structure. Coating thickness typically varies from 1 to 500 microns depending on the spray parameters employed.

The nanostructured titania feedstock is preferably thermally sprayed using HVOF, which is the preferred process for applying the feedstock since it produces highly uniform ceramic coatings. The spray parameters are carefully controlled so as to avoid a complete melting of the feedstock particles, which would lower the mechanical performance and biocompatibility of the coating.

The HVOF-sprayed titania coatings made from the nanostructured feedstock typically exhibits a bimodal microstructure, which is formed by controlling the heat input to the feedstock particles to produce a mixture of fully molten and semi-molten feedstock particles in the spray jet during thermal spraying. The percentage of semi-molten agglomerates can preferably vary from about 1 to about 50%.

The role of the fully molten nanostructured feedstock particles is to allow coating formation. Due to the lack of plasticity of ceramic materials (even at temperatures close to the melting point) it is necessary to have a degree of particle melting during thermal spraying in order to promote bonding and coating buildup. The semi-molten nanostructured feedstock particles retain part of the original nanostructure of the feedstock and are distributed throughout the coating, that is, they are (i) present at the coating-substrate interface, (ii) embedded within the coating and (iii) attached to the coating surface.

The semi-molten nanostructured feedstock particles situated at the coating-substrate interface enhance the bond strength of the coating. It has been observed that fully molten feedstock particles form gaps at the coating-substrate interface. However, semi-molten nanostructured feedstock particles exhibit a much reduced level of gap formation. The semi-molten nanostructured feedstock particles tend to increase interfacial toughness and help to arrest cracks that propagate at the interface, thereby increasing the bond strength of the coating.

The semi-molten nanostructured feedstock particles embedded in the coating microstructure (also called nanozones) enhance the coating toughness and the resistance to delamination. In conventional thermal spray ceramic coatings the long and well-defined splat boundaries provide easy crack propagation paths. In thermal spray ceramic coatings with bimodal microstructure, the splat boundary structure is periodically disrupted by the semi-molten nanostructured feedstock particles (nanozones). Cracks propagating through the splat boundaries tend to be arrested when reaching a nanozone, thereby enhancing coating toughness and its mechanical performance.

The semi-molten nanostructured feedstock particles (nanozones) attached to the surface of the coating are believed to play the role of promoting osteoblast growth and enhanced adhesion. Each nanozone on the coating surface exhibits the nanostructural characteristics necessary for vitronectin and fibronectin adsorption (i.e., nanoprotuberances, nanoirregularities and nanopores). Therefore the ability of these nanozones to selectively adsorb vitronectin and fibronectin provides anchors and/or centers for nucleation and proliferation of osteoblasts cells throughout the coating surface. It has been demonstrated by the results of osteoblast cell culture that the osteoblast cells attach and proliferate very well on the surface of HVOF-sprayed titania coatings made from a nanostructured feedstock.

The Vickers hardness (300 g) of an HVOF-sprayed titania made from a nanostructured feedstock deposited on a Ti-6A1-4V substrate was measured and found to be 824±40 (n=10). The Vickers hardness (300 g) of bulk (sintered) HA was measured (M. A. Lopes, F. J. Monteiro, J. D. Santos, “Glass-Reinforced Hydroxyapatite Composites: Fracture Toughness and Hardness Dependence on Microstructural Characteristics”, Biomaterials, 20 (1999) 2085-2090) and found to be 513±52. The HVOF-sprayed titania coating made from a nanostructured feedstock exhibits a cohesion strength (hardness) that is unmatched by the current HA bulk materials.

The Vickers hardness (100 g) of an HVOF-sprayed titania made from a nanostructured feedstock deposited on a Ti-6A1-4V substrate was measured and found to be 851±30 (n=10). The Vickers hardness values (100 g) of several plasma-sprayed HA coatings were measured (M. Espanol, V. Guipont, K. A. Khor, M. Jeandin, N. Llorca-Isem, “Effect of Heat Treatment on High Pressure Plasma Sprayed Hydroxyapatite Coatings”, Surface Engineering, Vol. 18, No. 3 (2002) 213-218) and the highest value found to be 275±40. The HVOF-sprayed titania coating made from a nanostructured feedstock exhibits a cohesion strength (hardness) that is unmatched by the current HA thermal spray coatings.

The adhesion of an HVOF-sprayed titania coating made from a nanostructured feedstock deposited on a Ti-6A1-4V substrate using a nanostructured feedstock was measured and found to be greater than 77 MPa (n=5) (testing method ASTM C633). The typical bond strength values of HA thermal spray coatings on Ti-6A1-4V substrates (testing method ASTM C633) are generally equal to or below 31 MPa (H. Li, K. A. Khor, P. Cheang, “Effect of Powders' Melting State on the Properties of HVOF Sprayed Hydroxyapatite Coatings”, Materials Science and Engineering A, 293 (2000) 71-80). The HVOF-sprayed titania coating made from a nanostructured feedstock exhibits very high mechanical integrity that is unmatched by current HA thermal spray coatings.

### EXAMPLE

A high velocity oxy-fuel (HVOF) torch was used to make TiO₂ nano coatings on a titanium alloy substrate. The following parameters were employed:

HVOF torch name: DJ2700-hybrid (Sulzer Metco, Westbury, N.Y., USA)

Propylene flow: 70 lpm (liters per minute)

Oxygen flow: 279 lpm (liters per minute)

Air flow: 202 lpm (liters per minute)

Carrier gas flow (Nitrogen—N2): 54 lpm (liters per minute)

Spray distance: 20 cm

Powder feed rate: 6 g/min.

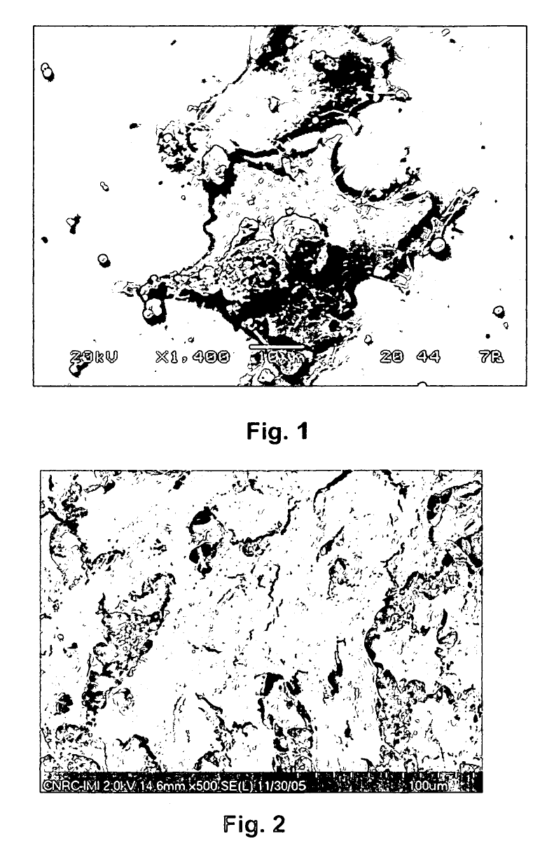

Human osteoblast cells were cultured in Dulbecco's modified Eagle medium (DMEM), supplemented with 10% fetal bovine serum and 0.5% antibiotics under standard conditions (37° C. and atmosphere 5% CO2) on the surface of titania coatings made in accordance with the above example for 3 days. FIG. 1 shows some cells attached to the coating surface.

Titania coatings made in accordance with an embodiment of the invention and air plasma sprayed HA coatings deposited on Ti-6A1-4V discs were steam autoclaved and put in the wells of a well plate. Osteoblast cells were isolated from the calvariae of 21-day Spargue Dawley rat fetuses by sequential collagenase digestion and plated in T-75 flasks in a growth medium (high glucose DMEM with 10% of fetal bovine serum (FBS)) as described by Bellows et al. (C. G. Bellows, J. E. Aubin, J. N. Heersche, M. E. Antosz, “Mineralized Bone Nodules Formed in vitro from Enzymatically Released Rat Calvaria Cell Populations”, Calcif. Tissue Int., 38 (1986) 143-154). After 24 h the attached cells were washed with phosphate buffer saline (PBS) and detached using 0.01% trypsin in PBS. The resuspended cells were counted and placed in the well culture plates, which contain implant disks, at 2×10⁴ cells/well in an osteoblast differentiation medium (ODM) (growth medium containing 50 mg of ascorbic acid/ml, 10 mM Na-b-glycerophosphate, and 1% antibiotics). The ODM was changed three times per week until endpoint, which was 7 days for SEM analysis and 15 days for alkaline phosphatase activity. The cells were incubated at 37° C. in a humidified atmosphere consisting of 95% air and 5% CO₂.

Scanning electron microscopy (SEM) observation after 7-day cell culture.

At the end of each incubation period, the cells were rinsed in phosphate buffer pH 7.2, fixed in a 0.089 M phosphate buffer solution containing 2.5% glutaraldehyde and 2.5 mM magnesium chloride, pH 7.2 for 3 hours. The samples were rinsed in 0.1 M phosphate buffer, postfixed in 1% osmium tetroxide for 1 h, washed in distilled water three times and then dehydrated in a graded series of ethanol solutions (70% through 100% dry ethanol). Specimens were then treated with mixtures consisting of 75:25, 50:50 and 25:75 ethanol:amyl acetate, and finally 100% amyl acetate. The samples were dried by the critical-point method, sputter-coated by platinum and observed using a field emission SEM (Hitachi S-4700, Hitachi Ltd., Tokyo, Japan) at an accelerating voltage of 2 kV to determine the adherence, morphology and growth of the osteoblasts on the different coatings.

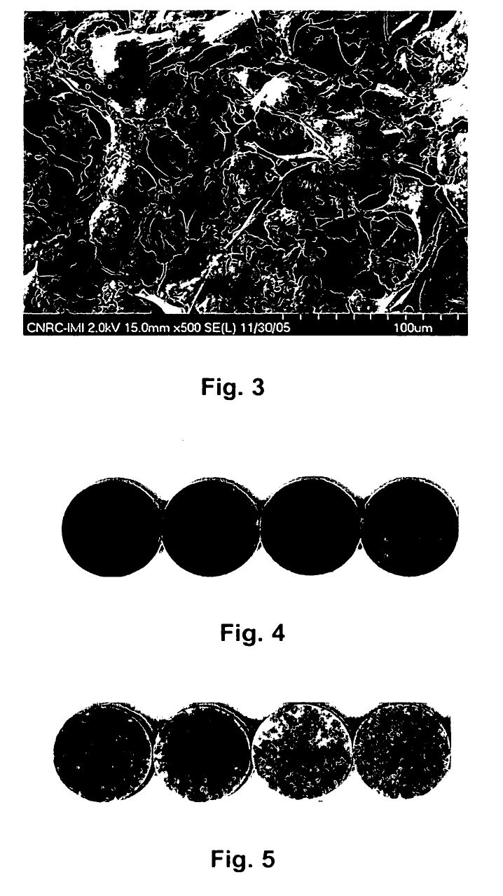

FIGS. 2 and 3 show the SEM analysis of the comparison of an osteoblast cell culture (obtained from rat calvaria) carried out during 7 days on the surface of a TiO₂ coating made in accordance with an embodiment of the invention and an air plasma sprayed HA coating (both coatings deposited on Ti-6A1-4V substrates). After a 7-day cell culture the osteoblast cells completely covered the surface of the TiO₂ coating, whereas, the surface of the HA coating was partially covered.

Alkaline phosphatase activity after 15-day cell culture.

The cell colonies were fixed and stained for alkaline phosphatase activity shown as a red stain over the coatings. To detect the alkaline phosphatase activity (ALP), which is the marker of mature osteoblast, cells were rinsed once with cold PBS, fixed in 10% cold neutral buffered formaldehyde for 15 minutes, rinsed with distilled water, and then left in distilled water for 15 minutes. Fresh solution (10 mg Naphtanol AS MX-PO₄ (Sigma-Aldrich Chemical Company, Oakville, ON, Canada) in 400 μl N,N-dimethylformamide, 50 ml distilled water, 50 ml Tris-HCl (0.2 M, pH 8.3), 60 mg red violet LB salt (Sigma Aldrich Chemical Company)) was placed into the wells of the 6-well plates, and incubated for 1 h at room temperature. The plates were rinsed with water, drained and air-dried, and then photographed. The ALP positive signal was quantified with Image J software (NIH, USA). For normalization, the background color was subtracted by setting a threshold. The percentage of the coating covered in red is a measure of the osteoprogenitor's ability to adhere, proliferate and differentiate toward the osteoblast lineage.

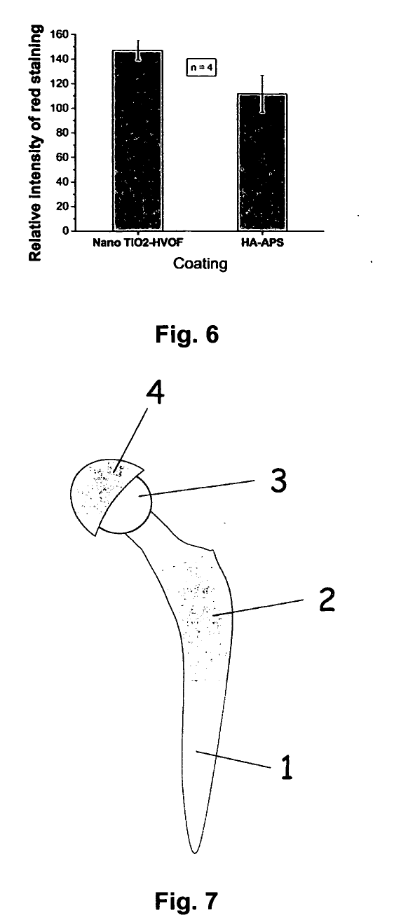

FIGS. 4 and 5 show the 15-day osteoblast cell culture (obtained from rat calvaria), stained in red as the result of the alkaline phosphatase activity, carried out on the surface of a TiO₂ coating made in accordance with an embodiment of the invention and an air plasma sprayed HA coating (both coatings deposited on Ti-6A1-4V substrates). FIG. 6 quantifies the relative intensity of red staining on the surface of the coatings measured from a threshold. The percentage of the coating covered in red is a measure of the osteoprogenitor's ability to adhere, proliferate and differentiate toward the osteoblast lineage.

The results shown in FIGS. 2 to 6 establish that the TiO₂ coating made in accordance with an embodiment of the invention exhibited a degree of cell proliferation and adhesion equivalent to or higher than that of air plasma sprayed HA coatings.

The invention can be applied to implants, such as hip joints, as shown in FIG. 7. In this figure, the hip joint comprises a stem 1 with an upper portion 2, a femoral head 3, and an acetabular cup 4. In this example, the upper portion 2 of the stem and the acetabular cup 4 are coated with a nanostructured titania coating in accordance with an embodiment of the invention.

Embodiments of the invention increase interfacial toughness and/or arrest crack formation and/or the formation of nanotexture on a surface area in a coating applied to a surface, such as the surface of a hip joint. The nano TiO₂ coatings in accordance with the invention can also be applied to other parts, in addition to the stem of hip-joints, including acetabular cups of hip-joints, artificial knee-joints, artificial teeth, and any type of implant to which conventional HA thermal spray coatings can be applied or bonding (osseointegration) of an implant is desired.

1. A method of making a biocompatible coating for an implant, comprising:
providing a feedstock of nanostructured agglomerated particles of a biocompatible material;
thermally spraying said particles onto a substrate to form a coating; and
controlling the spray parameters such that said agglomerated particles strike the substrate as a mix of fully molten and semi-molten particles and the semi-molten particles become distributed throughout the coating.

2. A method as claimed in claim 1, wherein the percentage of semi-molten particles varies in the coating from about 1 to about 50.

3. A method as claimed in claim 1, wherein said particles are nanostructured agglomerated titania particles.

4. A method as claimed in claim 1, wherein said particles are selected from the group consisting of: nanostructured hydroxyapatite, nanostructured zirconia, nanostructured alumina, and a combination thereof.

5. A method as claimed in claim 1, wherein the average temperature of the particles as they strike the substrate is 1500 to 3000° C.

6. A method as claimed in claim 1, wherein the average velocity of the sprayed particles lies in the range 100 to 1000 m/s.

7. A method as claimed in claim 1, wherein the coating thickness is between 1 and 500 microns.

8. A method as claimed in claim 1, wherein said particles are applied with a thermal spray torch.

9. A method as claimed in claim 7, wherein the thermal spray torch is a high velocity oxy-fuel (HVOF) torch.

10. A method as claimed in claim 7, wherein the spray torch is selected from the group consisting of: an air plasma spray (APS) torch, a vacuum plasma spray (VPS) torch, a low pressure plasma spray (PPS) torch, a high velocity air-fuel spray torch (HVAF), a high frequency pulse detonation (HFPD) torch, a detonation gun, a suspension plasma spray torch, and a suspension high velocity oxy-fuel spray torch.

11. A method as claimed in claim 8, wherein the thermal spray torch has a nozzle located about 1-100 cm from the substrate.

12. A method as claimed in claim 8, wherein the particles are supplied into the flow of the thermal spray torch at a rate lying in the range 1 to 100 g/min.

13. A method as claimed in claim 8, wherein the thermal spray torch is a plasma torch, and the plasma gases are selected from the group consisting of: argon, hydrogen, nitrogen, helium, and a combination thereof.

14. A method as claimed in claim 8, wherein the thermal spray torch is a combustion torch.

15. A method as claimed in claim 14, wherein the thermal spray torch is formed by the combustion of a mixture comprising oxygen or air as an oxidant and a combustible material selected from the group consisting of: propylene, hydrogen, methane, acetylene, propane, and other fuel gases compatible with thermal spray torches.

16. A method as claimed in claim 1, wherein said particles are sprayed on to the substrate for a time sufficient to build up a coating 1-500 microns thick.

17. A biocompatible implant comprising a substrate, and a thermally applied coating on said substrate for promoting osteoblast growth, said applied coating including a proportion of agglomerated nanostructured feedstock particles retaining their original nanostructure distributed throughout said applied coating.

18. A biocompatible implant as claimed in claim 17, wherein said coating is titania produced from a nanostructured feedstock.

19. A biocompatible implant as claimed in claim 17, wherein said coating is selected from the group consisting of: nanostructured hydroxyapatite, nanostructured zirconia, nanostructured alumina, and a combination thereof.

20. A biocompatible implant as claimed in claim 17, wherein the coating thickness is between 1 and 500 microns.

21. A biocompatible implant as claimed in claim 17, wherein said substrate is selected from the group consisting of: titanium, titanium alloys, CoCr alloys, stainless steel, polymer, ceramic or composite.

22. A biocompatible implant as claimed in claim 17, wherein said coating contains from about 1 to about 50% of said agglomerated feedstock particles retaining their original nanostructure.

23. A biocompatible implant as claimed in claim 17, wherein the nanostructural characteristics of said coating are formed from agglomerates having a diameter from about 0.1 to about 200 microns.

24. A biocompatible implant as claimed in claim 23, wherein said agglomerates are composed of particles smaller than 100 nm.

25. A biocompatible implant as claimed in claim 24, wherein said coating further comprises distributed individual nanoparticles with diameters varying from 100 to 300 nm.

26. A biocompatible implant as claimed in claim 17, wherein said implant is a component of an artificial hip joint, artificial knee joint, artificial tooth or any implant compatible with HA thermal spray coatings.

27. A biocompatible implant comprising a substrate, and a thermally applied coating on said substrate for promoting osteoblast growth, said applied coating including a proportion of agglomerated nanostructured titania particles retaining their original nanostructure distributed throughout said applied coating, and said titania particles being made of agglomerates having a diameter from about 0.1 to 200 microns, and with an individual particle size of up to 300 nm.
